(19) United States
(12) Patent Application Publication
(10) Pub. No.: US 2009/0062838 A1
(43) Pub. Date: Mar. 5, 2009

(54) SPIDER DEVICE WITH OCCLUSIVE BARRIER

(75) Inventors: John A. Brumleve, Bloomington, IN (US); Kurt J. Tekulve, Ellettsville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/845,452

(22) Filed: Aug. 27, 2007

(51) Int. Cl. A61B 17/00

(52) U.S. Cl. 606/198

(57) ABSTRACT

An occlusion device for occluding a body vessel. The occlusion device includes a first hub extending from a proximal end to a distal end and a tubular wall defining a lumen having a central axis. A plurality of arcuate legs are attached to the first hub and extend distally to a distal portion. The legs extend radially away from the central axis in an open configuration and extend substantially along the central axis in a closed configuration. A biocompatible material is attached to the plurality of legs to form an occlusive barrier when deployed within the body vessel. The biocompatible material either extends along and between each of the plurality of legs or it forms a disk attached to at least one leg.

### BACKGROUND

1. Field of the Invention

The present invention generally relates to vascular occlusion devices. More specifically, the invention relates to a spider shaped device with an occlusive barrier.

2. Description of Related Art

A number of different devices may be used to occlude a body cavity, for example, a blood vessel. When it is desirable to quickly occlude a blood vessel, an inflatable balloon may be used. However, balloon's have the disadvantage of being temporary. Another example of an occlusion device includes embolization coils. Embolization coils are permanent and promote blood clots or tissue growth over a period of time, thereby occluding the body cavity. In conjunction with the embolization coil, a spider shaped vascular obstruction device may be used to prevent dislodgment of the embolization coil while the blood clots or the tissue grows. A problem with this arrangement is that blood may continue to flow past the coil and spider device and through the body cavity until it finally occludes. It may take a significant period of time for sufficient tissue to grow to fully occlude the body cavity. This leaves a patient open to a risk of injury from the condition which requires the body cavity to be occluded. Also, since this arrangement is more complex since it requires the delivery of two separate devices to the vasculature.

In view of the above, it is apparent that there exists a need for an improved vascular occlusion device capable of occluding a body vessel quickly.

### SUMMARY

In satisfying the above need, as well as overcoming the enumerated drawbacks and other limitations of the related art, the present invention provides an occlusion device for occluding a body vessel. The occlusion device includes a first hub extending from a proximal end to a distal end along a central axis with a wall optionally defining a lumen. A first plurality of arcuate legs are attached to the first hub and extend distally to a distal portion. The legs extend radially away from the central axis in an open configuration and extend substantially along the central axis in a closed configuration. A biocompatible material is attached to the plurality of legs, forming an occlusive barrier when deployed within the body vessel. The biocompatible material may extend either along and between each of the first plurality of legs or form a disk attached to the distal portion of at least one of the first plurality of legs.

A third embodiment may optionally include a proximally extending member attached to the proximal end of the first hub. The proximally extending member includes a plurality of radially extending proximal fibers. The radially extending fibers may define, for example, a diameter less than a diameter of the body cavity. In another example, a distally extending member may be attached to the distal end of the first hub. The distally extending member may also include a plurality of radially extending distal fibers. In this example, the distally extending member and the distal fibers may distally extend beyond the disk.

In a fourth embodiment, a second plurality of arcuate legs may be added to the second embodiment. The second plurality of legs are attached to the proximal end of the first hub and extend proximally to a proximal section. The second plurality of legs open radially away from the central axis in an open configuration and lie substantially along the central axis in a closed configuration.

In a fifth embodiment, the second plurality of arcuate legs are added to a proximal end of a second hub and proximally extending to the proximal section. The second plurality of legs are attached at a connection point to the first plurality of legs and extend radially away from the central axis in an open configuration and lie substantially along the central axis in a closed configuration. In one example, a midpoint of the second plurality of legs are connected to a respective midpoint of the first plurality of legs.

In a sixth embodiment, the second plurality of arcuate legs are added to the proximal end of a second hub proximally extending to a proximal section. A distal end of the second hub is attached to the proximal end of the first hub by a connecting member. The second plurality of legs extend radially away from the central axis in an open configuration and extend substantially along the central axis in a closed configuration. In one example, the connecting member further comprises a plurality of circumferentially spaced arcuate members. A plurality of radially extending fibers may optionally be disposed within the volume defined by the arcuate members. In another example, the connecting member includes a plurality of radially extending fibers. The radially extending fibers may, for example, define a diameter less than a diameter of the body cavity.

A seventh embodiment includes the second plurality of arcuate legs attached to the proximal end of the second hub and proximally extend to a proximal section. The proximal end of the second hub is attached to the distal end of the first hub by a connecting member. The second plurality of legs extend radially away from the central axis in an open configuration and lie substantially along the central axis in a closed configuration. A length of the connecting member may be, for example, selected such that the distal end sections of the second plurality of legs oppose the distal portions of the first plurality of legs. In one example, the connecting member includes a plurality of radially extending fibers. The radially extending fibers may, for example, define a diameter less than a diameter of the body cavity.

In any of the above embodiments, the biocompatible material includes at least one of an extracellular matrix, biocompatible fibers, and mixtures thereof. For example, the extracellular matrix may include small intestine submucosa. In another example, the biocompatible fibers includes at least one of nylon, rayon, polyester, biocompatible polyurethanes, and mixtures thereof.

In yet another embodiment, the first hub includes a coupling member extending radially into the body lumen. In one example, the coupling member includes inner diameter threads. In another example the coupling member comprises an inwardly projecting flange.

The present invention also includes a delivery assembly for placing and retrieving any of the occlusion devices described above for occluding a body vessel. The assembly includes an outer sheath having a tubular body. The tubular body extends from a proximal part to a distal part and includes a lumen therethrough. The assembly also includes an inner member or catheter having proximal and distal portions. The inner catheter is disposed within the lumen of the outer sheath and configured for axial movement relative to the outer catheter. The occlusion device is coaxially disposed within the lumen of the outer catheter and is pushed distally by, or is removably coupled to, the distal portion of the inner catheter. The occlusion device is deployable through the distal part of the outer sheath by means of the relative axial movement of the inner catheter.

In one embodiment of the delivery assembly, the distal portion of the inner catheter includes a threaded section for engaging the coupling member of the occlusion device. In yet another example, the threaded section of the inner catheter includes a flexible threading coil.

The present invention also includes a method of occluding a body vessel having body walls. The method comprises providing one of the above occlusion devices within the body vessel. The method further includes positioning the device in a desired location to occlude the body vessel, opening the legs radially away from the central axis to expand the barrier within the body vessel, and coupling the occlusion device to the body walls of the body vessel.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1A is a partial section of a body vessel including an occlusion device in an open configuration according to a first embodiment of the present invention;

FIG. 1B is a partial section of a body vessel including an occlusion device in an open configuration according to a second embodiment of the present invention;

FIG. 2A is a partial section of the occlusion device of FIG. 1A collapsed within an outer sheath and coupled to an inner catheter of a delivery assembly;

FIG. 2B is a partial section of the delivery assembly of FIG. 2A showing one embodiment of a hub of the occlusion device coupled to the inner catheter;

FIG. 2C is a partial section of the delivery assembly of FIG. 2A showing another embodiment of the hub coupled to the inner catheter;

FIG. 3A is a side view of the occlusion device of FIG. 1B according to one example of a third embodiment of the present invention;

FIG. 3B is a side view of another example of the occlusion device of FIG. 3A;

FIG. 4A is a side view of the occlusion device of FIG. 1B according to a fourth embodiment of the present invention;

FIG. 4B is a side view of the occlusion device of FIG. 1B according to a fifth embodiment of the present invention;

FIG. 4C is a side view of the occlusion device of FIG. 1B according to one example of a sixth embodiment of the present invention;

FIG. 4D is a side view of another example of the occlusion device of FIG. 4C;

FIG. 5 is a side view of the occlusion device of FIG. 1B according to a seventh embodiment of the present invention;

FIG. 6A is a side view of one embodiment of a delivery and retrieval assembly for use with the occlusion device of the present invention;

FIG. 6B is an exploded view of the delivery and retrieval assembly of FIG. 6A; and

FIG. 7 is a flow-chart describing a method of occluding a body cavity using an occlusion device according to the present invention.

### DETAILED DESCRIPTION

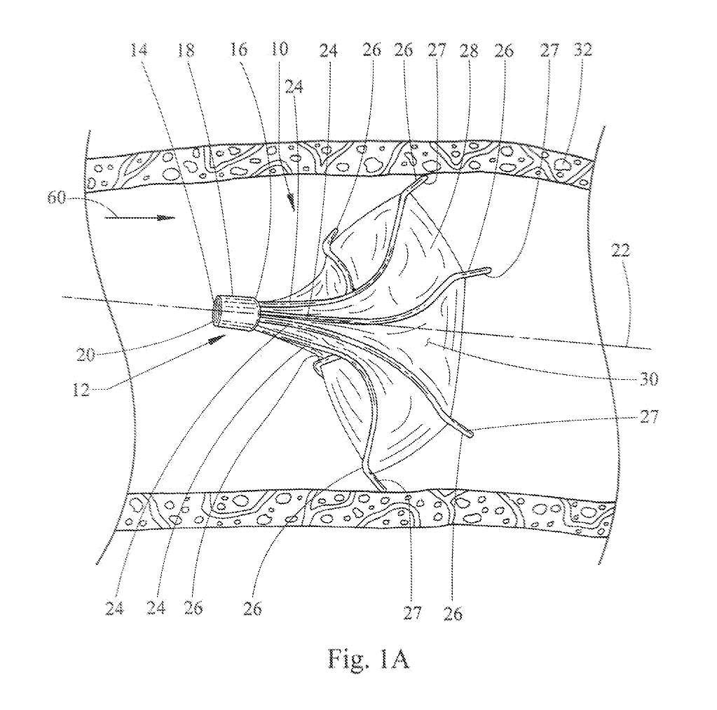

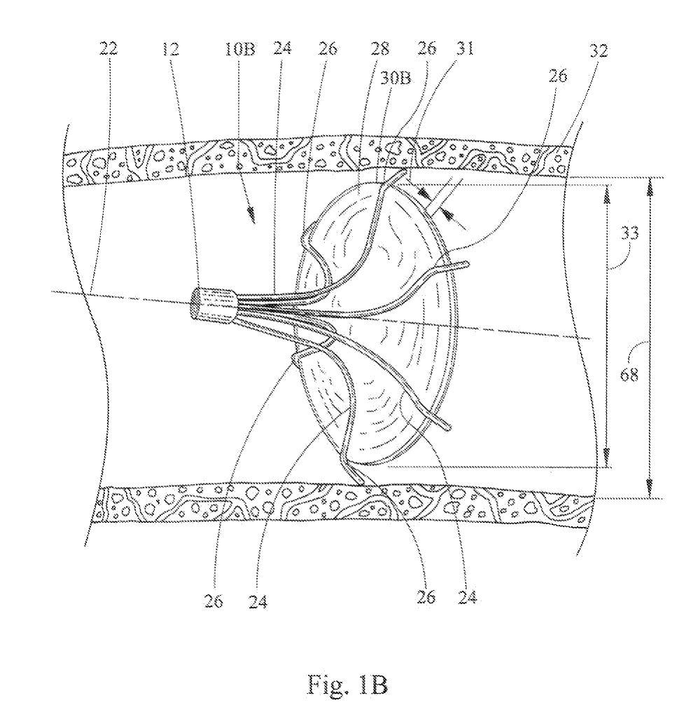

Referring now to FIG. 1A, a first embodiment of an occlusion device embodying the principles of the present invention is illustrated therein and designated at 10. As its primary components, the occlusion device 10 includes a first hub 12 extending from a proximal end 14 to a distal end 16 and including a wall 18 extending along a central axis 22 and optionally defining a lumen 20. A first plurality of arcuate legs 24 are attached to the first hub 12 and extend distally to a distal portion 26. A biocompatible material 28 is attached to the first plurality of legs 24 thereby forming an occlusive barrier 30 when deployed within a body vessel 32.

The first plurality of legs 24 are preferably attached to the distal end 16 and extend radially away from the central axis 22 when the device 10 is in an open configuration, for example, when deployed within the body vessel 32. While the exact number of the first plurality of legs 24 may vary depending on the needs of a particular application, the present example illustrates six legs. In other examples, the distal portion 26 of the legs may further include an angled distal end segment 27 for anchoring the device 10 to the body vessel 32. The distal end segment 27 may, for example, be angled back toward the central axis 22 to facilitate later removal of the device 10.

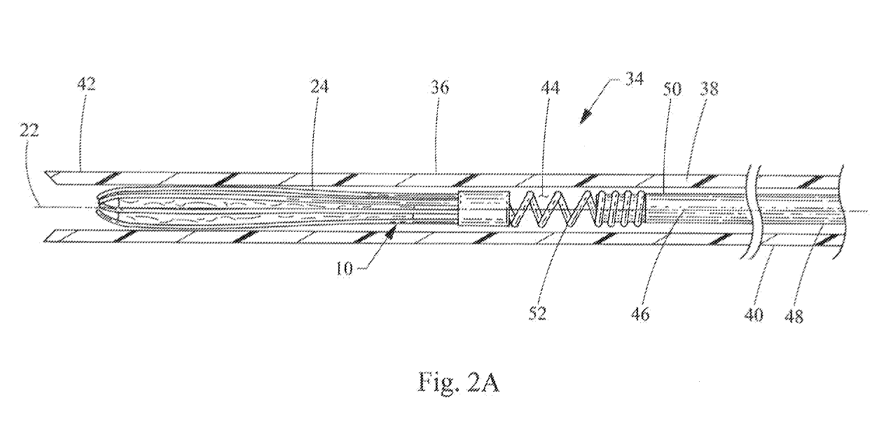

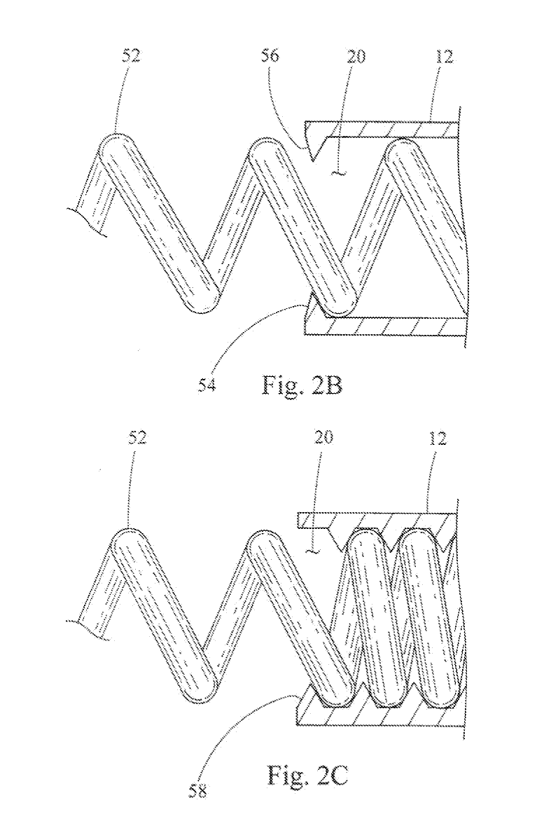

As best shown in FIG. 2A, the first plurality of legs 24 collapse into a closed configuration extending substantially along the central axis 22 when the device 10 is, for example, disposed within an outer sheath 36 of a delivery assembly 34. While the central axis 22 is shown as having a straight longitudinal path, it may also have other paths including, but not limited to, a curved path and a curled or spiral path depending, for example, on the shape of the body vessel 32 into which the device 10 is ultimately deployed. The outer sheath 36 has a tubular body 38 extending from a proximal part 40 to a distal part 42. An inner member 46 extending from a proximal portion 48 to a distal portion 50 is disposed within a sheath lumen 44 defined by the tubular body 38 and is configured for axial movement relative to the outer sheath 36. The inner member 46 may be any appropriate type of elongate pushing device including, for example, a catheter or stylet. The device 10 is pushed distally by, or removably coupled to, the distal portion 50 of the inner member 46 and deployable through the distal part 42 of the outer sheath 36 by means of the relative axial movement of the inner member 46.

The device 10 may be removably coupled by, for example, a threaded section 52 of the distal portion 50 of the inner member 46 engaging the first hub 12. In the example shown, the threaded section 52 includes a flexible threading coil. One non-limiting example of a threading coil is disclosed in U.S. Pat. No. 5,725,534 issued Mar. 10, 1998 which is herein incorporated by reference. Another non-limiting example of a threading coil is disclosed in U.S. Pat. No. 6,458,137 issued Oct. 1, 2002 which is herein incorporated by reference. As best shown in FIGS. 2B and 2C, the first hub 12 may include a coupling appendage 54. The coupling appendage 54 may be any complimentary feature appropriate for engaging the threaded section 52 of the inner catheter. For example, the coupling appendage 54 may project radially into the lumen 20 and include either an inwardly projecting flange 56 or inner diameter threads 58.

At least part of the device 10 may be made of any suitable material such as a superelastic material, stainless steel wire, cobalt-chromium-nickel-molybdenum-iron alloy, or cobalt-chrome alloy. It is understood that the device 10 may be formed of any suitable material that will result in a self-opening or self-expanding device 10, such as shape memory material. Shape memory materials or alloys have the desirable property of becoming rigid, i.e., returning to a remembered state, when heated above a transition temperature. A shape memory alloy suitable for the present invention is Ni—Ti available under the more commonly known name Nitinol. When this material is heated above the transition temperature, the material undergoes a phase transformation from martensite to austenite, such that material returns to its remembered state. The transition temperature is dependent on the relative proportions of the alloying elements Ni and Ti and the optional inclusion of alloying additives.

In one embodiment, the device 10 is made from Nitinol with a transition temperature that is slightly below normal body temperature of humans, which is about 98.6° F. Thus, when the device 10 is deployed in a body vessel and exposed to normal body temperature, the alloy of the device 10 will transform to austenite, that is, the remembered state, which for one embodiment of the present invention is the expanded state when the device 10 is deployed in the body vessel. To remove the device 10 it is cooled to transform the material to martensite which is more ductile than austenite, making the device 10 more malleable. As such, the device 10 can be more easily collapsed and pulled into a lumen of a catheter for removal.

In another embodiment, the device 10 is made from Nitinol with a transition temperature that is above normal body temperature of humans, which is about 98.6° F. Thus, when the device 10 is deployed in a body vessel and exposed to normal body temperature, the device 10 is in the martensitic state so that the device 10 is sufficiently ductile to bend or form into a desired shape, which for the present invention is the expanded state. To remove the device 10, the device 10 is heated to transform the alloy to austenite so that it becomes rigid and returns to a remembered state, which for the device 10 is a collapsed state.

Returning to the first embodiment of the device 10 shown in FIG. 1A, the biocompatible material 28 extends distally along and between the length of each of the first plurality of legs 24, approximately from the first hub 12 to the distal portion 26 of the legs 24 to form the barrier 30. In this example, it forms a thin web or membrane between each of the legs 24 and acts to occlude the body vessel 32 when the device 10 is deployed. When introduced into a body vessel 32, the device 10 may be oriented such that the first hub 12 is directed into a direction of blood flow as indicated by the arrow 60.

The barrier 30 includes other suitable materials configured to prevent blood, emboli and other fluids from flowing past, thereby occluding the body vessel 32. In one embodiment, the barrier 30 may be made of nylon, rayon, polyester, biocompatible polyurethanes, polytetrafluoroethylene (known as PTFE or under the trade name Teflon™), and mixtures thereof without falling beyond the scope or spirit of the present invention. In one example, the material may be made of one material and coated with another, such as the biocompatible polyurethane. In another example, the material may be made from the biocompatible polyurethane. In still another example, the barrier 30 may be made of connective tissue material including, for example, extracellular matrix (ECM).

One example of the biocompatible polyurethane is sold under the trade name THORALON (THORATEC, Pleasanton, Calif.). Descriptions of suitable biocompatible polyureaurethanes are described in U.S. Pat. Application Publication No. 2002/0065552 A1 and U.S. Pat. No. 4,675,361, both of which are herein incorporated by reference. Briefly, these publications describe a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. The present invention envisions the use of non-porous THORALON. Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes/polycarbamates and urea linkages (hereinafter “—C(O)N or CON type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible CON type polymers modified with cationic, anionic and aliphatic side chains may also be used. See, for example, U.S. Pat. No. 5,017,664. Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

As noted above, the barrier 30 may also be made of connective tissue material including, for example, extracellular matrix (ECM). As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In one particular embodiment, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. In many aspects, SIS is used to induce site-specific remodeling of both organs and tissues depending on the site of implantation. In practice, host cells are stimulated to proliferate and differentiate into site-specific connective tissue structures, which have been shown to completely replace the SIS material in time.

In another particular embodiment, the SIS may be used to temporarily adhere the barrier 30 to the walls of the body vessel 32 in which the device is deployed. SIS has a natural adherence or wetability to body fluids and connective cells comprising the connective tissue of a body vessel wall. Since it may be desirable to only temporarily occlude the body vessel 32, when the device 10 is deployed in the body vessel, host cells of the wall may adhere to the filter portion but will not differentiate, allowing for later retrieval of the device 10 from the body vessel 32. However, in other applications where permanent occlusion is desired the device 10 may remain in place and the host cells of the wall may differentiate into the barrier 30, eventually replacing the SIS and the barrier 30 with the host cells of the body vessel 32.

A second embodiment of the device 10 is shown in FIG. 1B and designated at 10B. In this embodiment, features of the device 10B common with the device 10 share common reference numbers. This embodiment is similar to the first embodiment except the biocompatible material 28 defines a disk shape barrier 30B, rather than a web or membrane extending between the legs 24. The disk barrier 30B is circular in shape and has a thickness 31 substantially less than a disk diameter 33. In the example shown, the thickness 31 is about twenty-five times less than the diameter 33. However, other examples may have any other appropriate relative dimensions depending on the needs of a particular application. The disk barrier 30B is attached to the distal portion 26 of at least one of the first plurality of legs 24. In this example, the disk barrier 30B is oriented substantially perpendicular to the central axis 22. However, it is also possible for the disk barrier 30B to be at an acute angle to the central axis 22 (not shown). In this case, the disk barrier 30B may be oval or elliptical in shape.

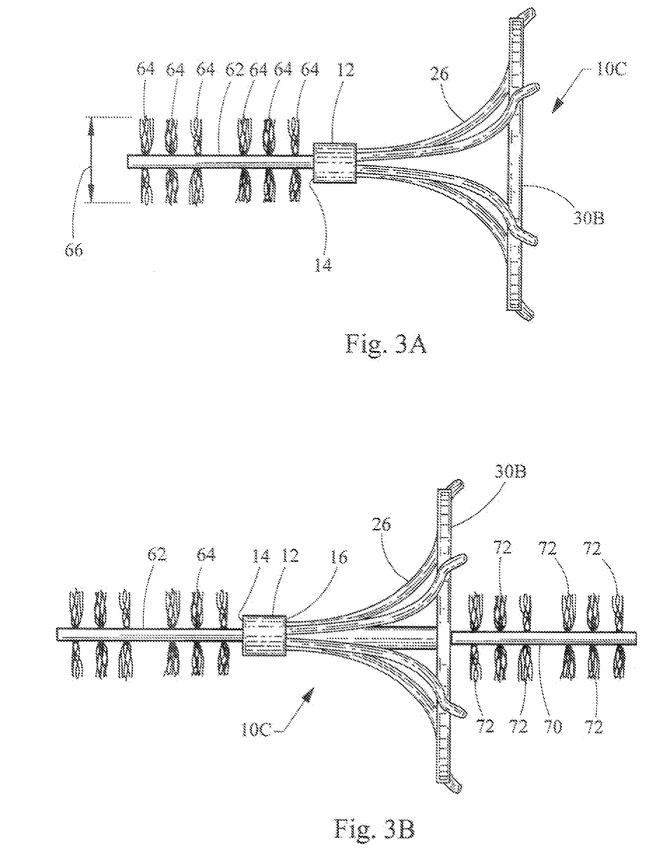

It should be noted that any of the embodiments described herein in FIGS. 3A-5 may incorporate either the disk barrier 30B, the thin web or membrane barrier 30 described with reference to FIG. 1A, or a combination of these barriers without falling beyond the scope of the present invention.

Two examples of a third embodiment of the device 10 are shown in FIGS. 3A and 3B and designated at 10C. As above, features of the device 10C common with the device 10 share common reference numbers. This embodiment begins with the embodiment of the device 10 or 10B of FIGS. 1A and 1B, and adds a proximally extending member 62 attached to the proximal end 14 of the first hub 12. In the example shown, the proximally extending member 62 includes a plurality of radially extending proximal fibers 64. It should be noted that in any of the other embodiments described herein, with particular reference to FIGS. 3A, 3B, 4C, 4D and 5, the radially extending fibers 64 may either define a diameter 66 less than a diameter 68 of the body vessel 32 (see FIG. 1B), or the diameter 66 may be greater than the diameter 68. Another example of the present embodiment may further include a distally extending member 70 having a plurality of radially extending distal fibers 72 attached to the distal end 16 of the first hub 12 and extending distally through the disk barrier 30B. Optionally, as noted above, the disk barrier 30B may be replaced the thin web or membrane barrier 30 of FIG. 1A (not shown). The radially extending fibers 64 and 72 may be any of the biocompatible materials described above. In a preferred embodiment, the radially extending fibers 64 and 72 may be polyester fibers.

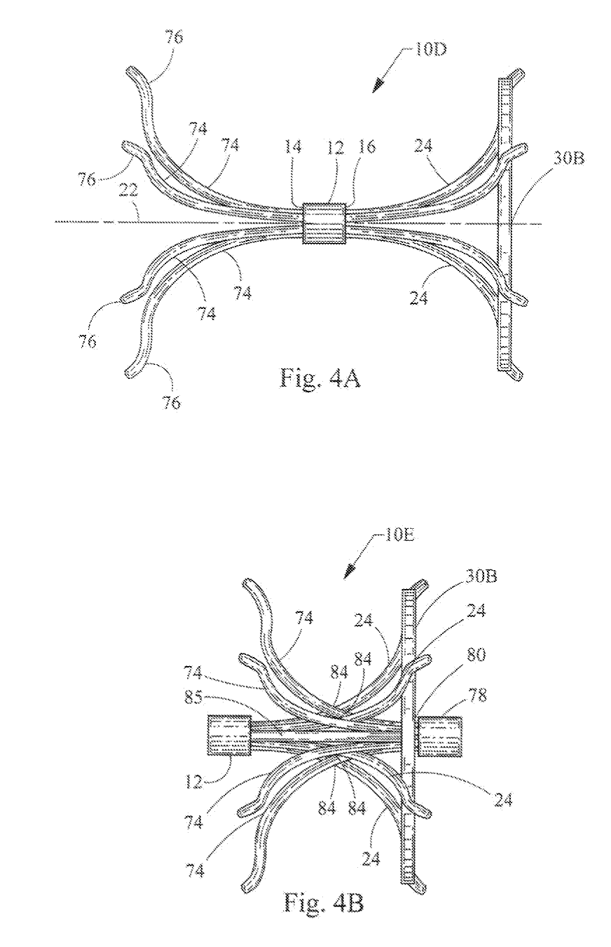

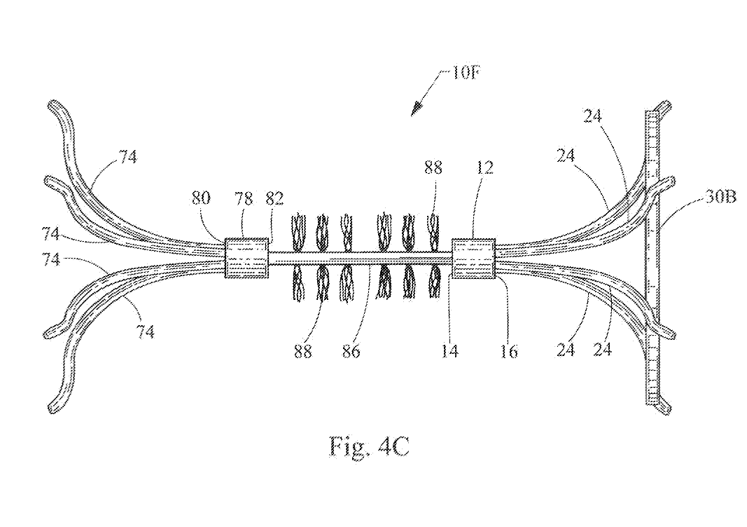

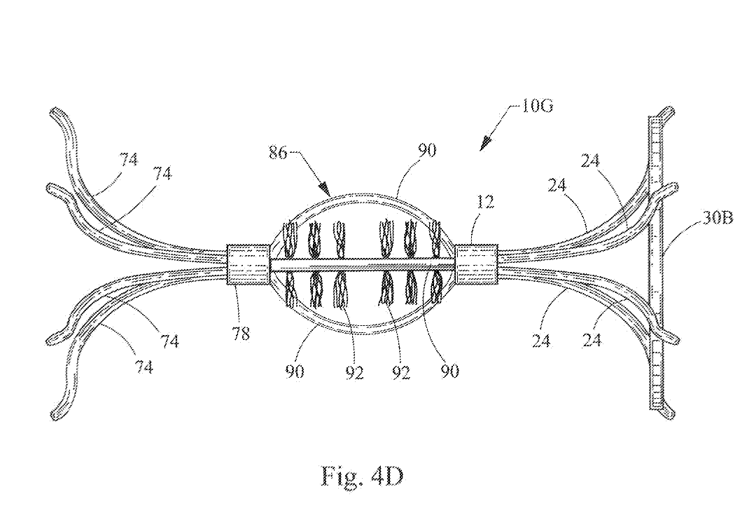

A fourth embodiment of the device 10 is shown in FIG. 4A and designated at 10D. As above, features of the device 10D common with the device 10 share common reference numbers. This embodiment begins with the embodiment of the device 10 or 10B of FIGS. 1A and 1B, and adds a second plurality of arcuate legs 74 attached to the proximal end 14 of the first hub 12 and extending in a proximal direction to a proximal section 76. The second plurality of legs 74 are substantially the same as the first plurality of legs 24 wherein they extend radially away from the central axis 22 in an open configuration and extend substantially along the central axis 22 in a closed configuration.

A fifth embodiment of the device 10 is shown in FIG. 4B and designated at 10E. As above, features of the device 10E common with the device 10 share common reference numbers. This embodiment begins with the embodiment of the device 10 or 10B of FIGS. 1A and 1B, and adds the second plurality of legs 74 described above with reference to FIG. 4A. However, in this embodiment the second plurality of legs 74 are attached to a proximal end 80 of a second hub 78 and are also attached to the first plurality of legs 24 at a plurality of connection points 84. In one example, the connection points 84 may be at the respective mid points of the first and second plurality of legs 24 and 74. In another instance, a middle member 85 may extend between the hubs 12 and 78. The middle member 85 may include, for example, a coiled wire, a cut cannula, a solid wire, and a tube.

A sixth embodiment of the device 10 is shown in FIG. 4C and designated at 10F. As above, features of the device 10F common with the device 10 share common reference numbers. This embodiment begins with the embodiment of the device 10C as shown in FIG. 3A and adds the second plurality of legs 74 to the proximal end 80 of the second hub 78 as described above with reference to FIG. 4B. However, in this embodiment a distal end 82 of the second hub 78 is attached to the proximal end 16 of the first hub 12 by a connecting member 86. The connecting member 86 may include, for example, a coiled wire, a cut cannula, a solid wire, and a tube. This example may also include a plurality of radially extending fibers 88 disposed on the connecting member 86 similar to those described above. Another example of the present embodiment is shown in FIG. 4D and designated at 10G. In this example, the connecting member 86 is formed from a plurality of circumferentially spaced arcuate members 90. A plurality of radially extending fibers 92 may optionally be disposed within the volume defined by the plurality of arcuate members 90.

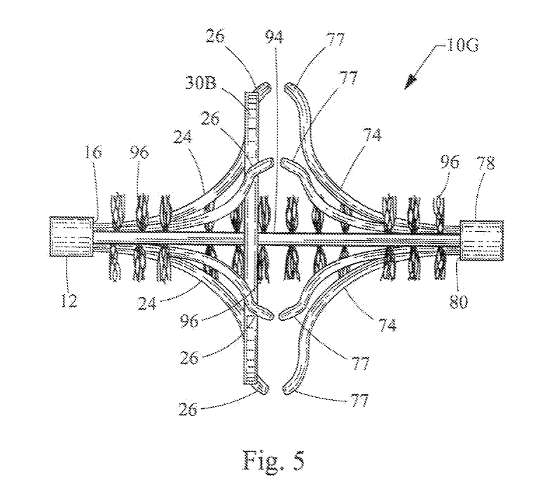

A seventh embodiment of the device 10 is shown in FIG. 5 and designated at 10G. As above, features of the device 10G common with the device 10 share common reference numbers. This embodiment begins with the embodiment of the device 10 or 10B of FIGS. 1A and 1B, and adds the second plurality of legs 74 attached to the proximal end 80 of a second hub 78 as described above with reference to FIG. 4B. However, in this embodiment the proximal end 80 of the second hub 78 is connected to the distal end 16 of the first hub 12 by a connecting member 94. A length of the connecting member 94 is selected such that distal sections 77 of the second plurality of legs 74 oppose the distal portions 26 of the first plurality of legs 24. The connecting member 94 may include, for example, a coiled wire, a cut cannula, a solid wire, and a tube. One example may include a plurality of radially extending fibers 96 disposed on the connecting member 94.

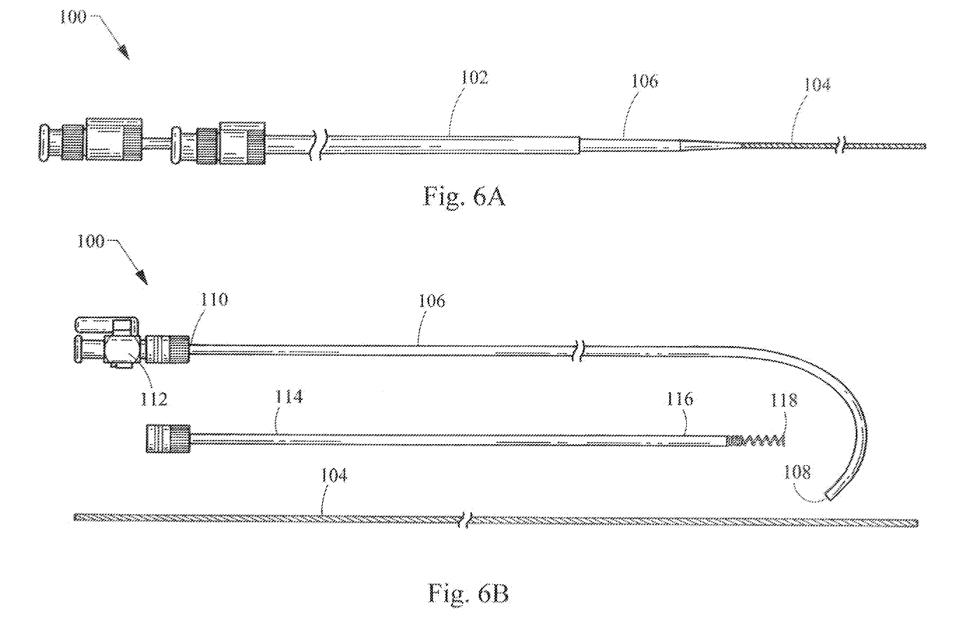

FIGS. 6A and 6B depict a delivery assembly 100 for introducing and retrieving the occlusion device for occluding a body vessel in accordance with another embodiment of the present invention. As shown, the delivery assembly 100 includes a polytetrafluoroethylene (PTFE) introducer sheath 102 for percutaneously introducing an outer sheath 106 (equivalent to the outer sheath 36 described above) into a body vessel. Of course, any other suitable material for the introducer sheath 102 may be used without falling beyond the scope or spirit of the present invention. The introducer sheath 102 may have any suitable size, for example, between about three-french to eight-french. The introducer sheath 102 serves to allow the outer sheath 106 and an inner member, stylet or catheter 114 to be percutaneously inserted to a desired location in the body vessel. The introducer sheath 102 receives the outer sheath 106 and provides stability to the outer sheath 106 at a desired location of the body vessel. For example, the introducer sheath 102 is held stationary within a common visceral artery, and adds stability to the outer sheath 106, as the outer sheath 106 is advanced through the introducer sheath 102 to a occlusion area in the vasculature.

As shown, the assembly 100 may also include a wire guide 104 configured to be percutaneously inserted within the vasculature to guide the outer sheath 106 to the occlusion area. The wire guide 104 provides the outer sheath 106 with a path to follow as it is advanced within the body vessel. The size of the wire guide 104 is based on the inside diameter of the outer sheath 106 and the diameter of the target body vessel.

When a distal end 108 of the outer sheath 106 is at the desired location in the body vessel, the wire guide 104 is removed and the occlusion device, having a proximal segment releasably coupled to a distal portion 116 of the inner catheter 114, is inserted into the outer sheath 106. The inner catheter 114 is advanced through the outer sheath 106 for deployment of the device through the distal end 108 to occlude the body vessel during treatment of, for example, an aneurism. In this example, the distal portion 116 is shown including a flexible threading coil 118 (similar to the threaded section 52 described above).

The outer sheath 106 further has a proximal end 110 and a hub 112 to receive the inner catheter 114 and device to be advanced therethrough. The size of the outer sheath 106 is based on the size of the body vessel in which it percutaneously inserts, and the size of the device.

In this embodiment, the device and inner catheter 114 are coaxially advanced through the outer sheath 106, following removal of the wire guide 104, in order to position the device to occlude the body vessel. The device is guided through the outer sheath 106 by the inner catheter 114, preferably from the hub 112, and exits from the distal end 108 of the outer sheath 106 at a location within the vasculature where occlusion is desired.

Likewise, this embodiment may also retrieve the device by positioning the distal end 108 of the outer sheath 106 adjacent the deployed device in the vasculature. The inner catheter 114 is advanced through the outer sheath 106 until the distal portion 116 protrudes from the distal end 108 of the outer sheath 106. The distal portion 116 is coupled to a proximal end of the device, after which the inner catheter 114 is retracted proximally, drawing the device into the outer sheath 106.

It is understood that the assembly described above is merely one example of an assembly that may be used to deploy the occlusion device in a body vessel. Of course, other apparatus, assemblies and systems may be used to deploy any embodiment of the occlusion device without falling beyond the scope or spirit of the present invention.

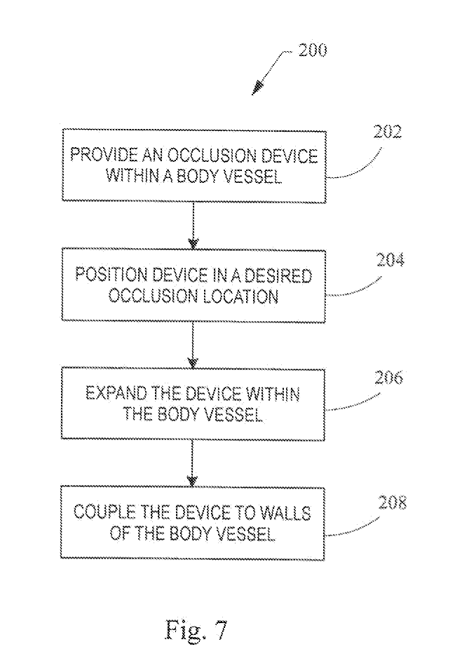

Turning to FIG. 7, a flow chart designated at 200 is provided describing a method for occluding a body vessel such as a blood vessel. The method includes providing any of the above occlusion devices within the body vessel at box 202. Box 204 includes positioning the occlusion device in a desired location to occlude the body vessel. Box 206 includes expanding the occlusion device within the body vessel and box 208 includes coupling the occlusion device to walls of the body vessel.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

I claim:

1. An occlusion device for occluding a body vessel, the occlusion device comprising:
a first hub extending from a proximal end to a distal end and along a central axis;
a first plurality of arcuate legs distally extending from the first hub to a distal portion, the legs extending radially away from the central axis in an open configuration and extending substantially along the central axis in a closed configuration; and
a biocompatible material being attached to the plurality of legs and forming a barrier when deployed within the body vessel, the biocompatible material either extending along and between each of the first plurality of legs or forming a disk attached to at least one of the first plurality of legs.

2. The occlusion device of claim 1 further comprising a proximally extending member attached to the first hub, the proximally extending member including a plurality of radially extending proximal fibers.

3. The occlusion device of claim 2 further comprising a distally extending member attached to the first hub, the distally extending member including a plurality of radially extending distal fibers.

4. The occlusion device of claim 3 wherein the distally extending member and distal fibers extends beyond the biocompatible material.

5. The occlusion device of claim 1 further comprising:
the first plurality of arcuate legs being attached to the distal end of the first hub and a second plurality of arcuate legs being attached to the proximal end and proximally extending to a proximal section, the second plurality of legs extending radially away from the central axis in an open configuration and extending substantially along the central axis in a closed configuration.

6. The occlusion device of claim 1 further comprising:
a second plurality of arcuate legs being attached to a second hub and proximally extending to a proximal section, the second plurality of legs being attached to the first plurality of legs at connection points and extending radially away from the central axis in an open configuration and extending substantially along the central axis in a closed configuration.

7. The occlusion device of claim 6 wherein the connection points may be at respective midpoints of the first and second plurality of legs.

8. The occlusion device of claim 1 further comprising:
a second plurality of arcuate legs being attached to a second hub and proximally extending to a proximal section, a distal end of the second hub being attached to the proximal end of the first hub by a connecting member, and the second plurality of legs extending radially away from the central axis in an open configuration and extending substantially along the central axis in a closed configuration.

9. The occlusion device of claim 8 wherein the connecting member further comprises a plurality of circumferentially spaced arcuate members.

10. The occlusion device of claim 9 wherein a plurality of radially extending fibers are disposed within a volume defined by the arcuate members.

11. The occlusion device of claim 8 wherein the connecting member includes a plurality of radially extending fibers.

12. The occlusion device of claim 1 further comprising:
a second plurality of arcuate legs being attached to a second hub and proximally extending to a proximal section, a proximal end of the second hub being attached to the distal end of the first hub by a connecting member, the second plurality of legs extending radially away from the central axis in an open configuration and extending substantially along the central axis in a closed configuration, a length of the connecting member being selected such that the distal sections of the second plurality of legs oppose the distal portions of the first plurality of legs.

13. The occlusion device of claim 12 wherein the connecting member includes a plurality of radially extending fibers.

14. The occlusion device of claim 1 wherein a distal portion of the legs include a distal end segment angled back toward the central axis.

15. The occlusion device of claim 1 wherein the biocompatible material includes an extracellular matrix.

16. The occlusion device of claim 15 wherein the extracellular matrix includes small intestine submucosa.

17. The occlusion device of claim 1 wherein the biocompatible material includes at least one of nylon, rayon, polyester, polytetrafluoroethylene, biocompatible polyurethanes, and mixtures thereof.

18. The occlusion device of claim 1 wherein first hub includes a wall defining a first lumen extending along the central axis.

19. The occlusion device of claim 18 wherein a coupling appendage extends from the wall into the first lumen.

20. The occlusion device of claim 19 wherein the coupling appendage comprises an inwardly projecting flange.

21. The occlusion device of claim 1 wherein the plurality of legs includes at least six legs.

22. A delivery assembly for placing and retrieving an occlusion device for occluding a body vessel, the assembly comprising:
an outer sheath having a tubular body extending from a proximal part to a distal part and the tubular body including a sheath lumen extending therethrough;
an inner member extending from a proximal portion to a distal portion, the inner member being disposed within the sheath lumen and configured for axial movement relative to the outer sheath;
the occlusion device being coaxially disposed within the sheath lumen and removably coupled to the distal portion of the inner member and deployable through the distal part of the outer sheath by means of the relative axial movement of the inner member, the occlusion device comprising:
at least one hub extending from a proximal end to a distal end and including a tubular wall defining a lumen having a central axis and a coupling appendage, the coupling member being in engagement with the distal portion of the inner catheter;
at least one plurality of arcuate legs being attached to the hub and extending distally to a distal portion, the legs extending radially away from the central axis in an open configuration and extending substantially along the central axis in a closed configuration; and
a biocompatible material being attached to the plurality of legs thereby forming at least one barrier when deployed within the body vessel.

23. The delivery assembly of claim 22 wherein the biocompatible material includes small intestine submucosa.

24. The delivery assembly of claim 22 wherein the biocompatible material includes at least one of nylon, rayon, polyester, polytetrafluoroethylene, biocompatible polyurethanes, and mixtures thereof.

25. The delivery assembly of claim 22 wherein the distal portion of the inner member includes a threaded section.

26. The delivery assembly of claim 22 wherein the coupling member includes an inwardly projecting flange.

27. A method of occluding a body vessel having body walls, the method comprising:
providing an occlusion device within the body vessel, the device having a hub extending from a proximal end to a distal end and including a tubular wall defining a lumen and having a central axis, a plurality of distally extending arcuate legs being attached to the hub, a biocompatible material being attached to the plurality of legs and forming a barrier;
positioning the device in a desired location to occlude the body vessel;
opening the legs radially away from the central axis to expand the barrier within the body vessel;
coupling the occlusion device to the body walls of the body vessel.
